(19) United States
(12) Patent Application Publication
(10) Pub. No.: US 2004/0038373 A1
(43) Pub. Date: Feb. 26, 2004

(54) Apoptotic EBV-transformed lymphocytes, a therapeutic agent for post-transplant lymphoproliferative disorder

(76) Inventors: Matthew S. Platz, Columbus, OH (US); Michael Caligiuri, Upper Arlington, OH (US); Susan Olesik, Dublin, OH (US); Helene Balladur, Columbus, OH (US); Jacqueline S. Ward, Columbus, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/314,869

(22) Filed: Dec. 9, 2002

Related U.S. Application Data
(60) Provisional application No. 60/338,411, filed on Dec. 7, 2001.

(51) Int. Cl. C12N 15/86; C12N 7/00

(52) U.S. Cl. 435/235.1; 435/239; 435/456

(57) ABSTRACT

Cell preparations comprising a plurality of apoptotic EBV-transformed B lymphocytes, and methods of producing cell preparations comprising a plurality of apoptotic EBV-transformed B lymphocytes are provided. The methods comprise transforming B lymphocytes with EBV, incubating the transformed B lymphocytes with a flavin photosensitizer, such as riboflavin or a lumichrome-resistant photosenstizer, adding a non-toxic anti-oxidant, and exposing the lymphocytes to photoradiation of an appropriate wavelength to activate the photosensitizer. Also provided are methods of using the apoptotic EBV-transformed B lymphocyte cell preparations to elicit production of EBV-specific T cells in human patients. Finally, methods of treating organ transplant patients comprising administering an effective amount of the apoptotic EBV-transformed B-lymphocytes cell preparation to the patients prior to transplantation are provided.

This application claims priority from U.S. Provisional Patent Application Serial No. 60/338,411, which was filed on Dec. 7, 2001, the entirety of which is incorporated herein by reference.

### BACKGROUND

Epstein-Barr virus is a gamma herpes virus that resides in approximately 85% of adults in the United States. EBV specifically infects human B cells, which in cell culture or in an immune compromised host, will transform to a malignant phenotype and grow without control (i.e., immortalize). Most people acquire EBV sub-clinically, but for some, the initial infection is heralded by infectious mononucleosis. Subsequently, a normal, healthy adult harbors few EBV+ B lymphocytes in the body (so called “latently infected”), and an “army” of EBV-specific T cells that keep the EBV+ B cell “in check” from ever reactivating and causing EBV+ B cell lymphoma in the human for the rest of their life. However, if an individual has suppression of their T lymphocytes, for whatever reason (e.g., congenital, acquired, or iatrogenic immune deficiency that follows solid organ transplantation), endogenous reactivation of the latently infected B cells by the lytic form of EBV can be fatal.¹ Further, in immune suppressed children who have yet to be exposed to EBV, primary infection by the virus during states of iatrogenic immune deficiency such as occurs with immune suppressive therapy for solid organ transplantation is highly fatal. Indeed, 20% of children who undergo liver transplantation die from this complication (i.e., post-transplant lymphoproliferative disorder, or PTLD).¹

PTLD is highly fatal in children undergoing solid organ transplantation as noted above, and can complicate approximately 2% of adult patients undergoing kidney transplantation and up to 20% of cardiac transplants in adults.¹ These patients must take immunosuppressive therapy so they do not reject their transplanted organs. This therapy suppresses T cells that guard against either primary EBV infection or re-infection from latent EBV. PTLD is fatal in approximately 30-50% of cases. For the vast majority of adults, treatment of PTLD consists of reduction in immune suppressive therapy, sometimes followed by immunotherapy or chemotherapy. Reduction in immune therapy is an option in adult transplant patients because adults are previously exposed to EBV and therefore have immunologic memory T cells that can be activated against PTLD once immune suppression is reduced. However, the vast majority of children do not have EBV specific T cells, and in childhood solid organ transplants, the lack of a prior infection/exposure of EBV can lead to a rapid, often fatal complication from EBV-associated PTLD, as noted above. Indeed, some transplantation centers will not allow liver transplantation if a child does not have prior exposure to EBV.

An effective single peptide vaccine for EBV has yet to be developed. Thus, there is a need for compositions and methods which can be used to protect human subjects, particularly children, who are about to undergo an organ transplant against PTLD.

### SUMMARY OF THE INVENTION

The present invention provides compositions and methods which use such compositions to prophylactically protect a human subject about to undergo an organ transplant against post-transplant lymphoproliferative disorder (PTLD). In one embodiment the composition is a cell preparation which comprises a plurality of apoptotic EBV transformed B lymphocytes. Such lymphocytes comprise flavin-DNA adducts. The cell preparation may further comprise an adjuvant. In certain embodiments, the method comprises administering a therapeutically effective amount of this cell preparation or the isolated lymphocytes contained therein to the patient.

In another aspect the method comprises a method of producing a cell preparation which comprises a plurality of apoptotic EBV transformed lymphocytes. The method comprises: transforming B lymphocytes with EBV, incubating said EBV-transformed B lymphocytes in a medium comprising the flavin photosensitizer riboflavin or another flavin photosensitizer, referred to hereinafter as the “LC-resistant photosensitizer” under conditions which permit uptake of the photosensitizer by the lymphocytes, adding a non-toxic antioxidant to the medium; and exposing the cells to photoradiation of an appropriate wavelength to activate the riboflavin or the LC-resistant flavin photosensitizer. Preferably, at least 25% of the EBV-transformed lymphocytes are in S phase when incubated with the riboflavin; more preferably at least 35% of the EBV-transformed lymphocytes are in the S phase when incubated with the riboflavin; and even more preferably, at least 50% of the EBV-transformed lymphocytes are in the S phase when incubated with the riboflavin. Preferably, the wavelength of light used to activate the the riboflavin or LC-resistant flavin photosensitizer is in the visible region, i.e., from about 400 to about 700 nm; more preferably, the wavelength is in the range from about 400 to about 500 nm.

The present invention also relates to the cell preparation and the apoptotic cells produced by the present method. Such apoptotic cells comprise flavin-DNA adducts.

In another aspect the method comprises eliciting an EBV-specific immune response in vitro or in vivo. The method comprises contacting lymphocytes, particularly T lymphocytes with the apoptotic EBV-transformed B lymphocytes of the present invention. The EBV-transformed B lymphocytes may be purified or partially purified prior to application.

In another aspect, the invention comprises a method of treating an organ transplant patient, particularly someone who is about to undergo an organ transplant, and has little or no circulating levels of EBV-specific T cells. This method is particularly suitable for children who are about to undergo organ transplants. The method comprises administering the present cell preparation in an amount sufficient to elicit production of EBV-specific T-cells in the patient. The cell preparation is preferably administered prior to transplantation and prior to administration of immunosuppressive drugs. Preferably, the cell preparation is administered in an amount sufficient to retard, prevent, or reduce development of post-transplant lymphoproliferative disorder in the patient.

### BRIEF DESCRIPTION OF THE DRAWINGS

FIG. 1 is a Jablonski diagram.

FIG. 2 is a graph of Riboflavin Efficiency showing the percentage of cells dead after 24 hours versus the concentration of riboflavin used.

FIG. 3 is a schematic representation of the four stages of the cell cycle.

FIG. 4 is a graph of the Percentage of Cells in the S-Phase versus Time After Aphidicolin Treatment.

FIG. 5 is a density plot illustrating the assay for apoptosis.

FIG. 6 is a density plot illustrating the apoptosis assay results for unsynchronized cells, synchronized cells, and a control.

### DETAILED DESCRIPTION OF THE INVENTION

Methods of Producing Apoptotic EBV-Transformed Lymphocytes

In one aspect, the present invention provides a method of producing apoptotic EBV-transformed B lymphocytes. Such method comprises infecting B lymphocytes that have been obtained from a subject with EBV, incubating the EBV transformed B lymphocytes with riboflavin or an LC-resistant flavin photosensitizer under conditions which permit the riboflavin or the LC-resistant flavin photosensitizer to enter the B lymphocyte and bind to nucleic acid molecules in the cell, adding a non-toxic anti-oxidant to the incubation medium, and exposing the cells to photoradiation of an appropriate wavelength to activate the riboflavin or the LC-resistant photosensitizer.

A. Infection and Transformation of Lymphocytes with EBV.

Lymphocytes are obtained from a human subject, preferably from a human subject who is about to undergo an organ transplant, using techniques known in the art. Although not necessary, the lymphocytes may be separated into specific B cell and T cell populations prior to exposure to EBV. The B lymphocytes which contain receptors for EBV are then infected with EBV using methods known in the art and maintained in culture. In the infected B lymphocyte, the EBV genome is replicated by cellular DNA polymerase during S phase and persists as multiple extrachromosomal double-stranded DNA EBV episomes. EBV episomes are also known to integrate into chromosomal DNA in latently infected cells. When grown in vitro, the EBV infected B lymphocytes undergo transformation. Such EBV transformed B lymphocytes comprise EBV-specific antigens which can be detected with antisera.

B. Riboflavin Activation

Riboflavin (RB) is a component of the B2 vitamin complex and is present in aerobic organisms. It is the precursor of flavin mononucleotide (FMN) and flavin adenine dinucleotide (FAD).³

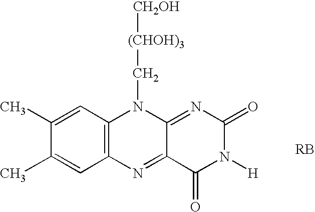

Riboflavin is not retained by the human body and is a required component of a healthy diet. It is present in milk, beer, eggs, yeast, and leafy vegetables. Riboflavin absorbs strongly in both the UV and visible regions of the spectrum with maxima at 220, 265, 375, and 446 nm and has a yellow-orange color.^{3,4} Exposure of RB in foodstuffs to sunlight converts the vitamin to lumichrome (LC) which is also a metabolic break down product of riboflavin in the human body.³ Lumiflavin (LF) is formed upon photolysis of RB in alkaline solutions.^{4,5}

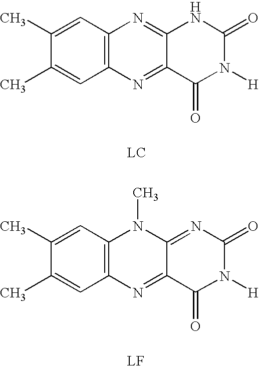

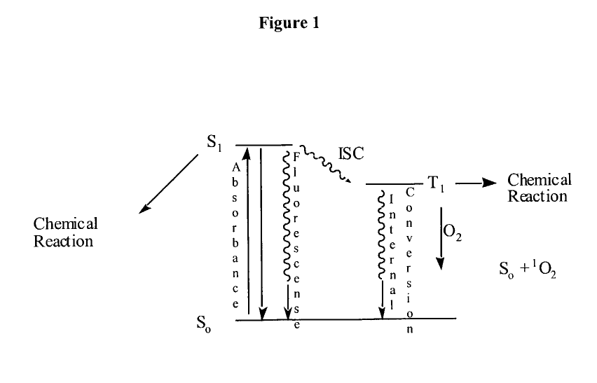

Riboflavin acts as a photosensitizer. Photosensitizer photophysics and photochemistry can be usefully summarized with the aid of the Jablonski diagram, shown in FIG. 1.^{15,16} The sensitizer in its ground electronic state is referred to as S_{O}. Upon absorption of light it is converted to an electronically excited state which in condensed phase immediately (<<10⁻¹¹ s) relaxes to the lowest vibrational level of the lowest excited state (S₁). The lifetimes of S₁ states in solution are usually in the range of 1-10 ns and are controlled by internal conversion (IC) and fluorescence (F) decay back to S_{O}, by intersystem crossing (ISC) to a paramagnetic triplet state (T₁) and by inter and intramolecular chemical reactions. Because S₁ is short-lived, bimolecular reactions of S₁ will be inefficient unless the trapping agent is rather concentrated (0.1-1.0 M) or the sensitizer and the trap are complexed. A sensitizer bound to protein or nucleic acid will likely react in its S₁ state. ^{15,16} Common reactions of S₁ are electron transfer and cycloaddition. Fluorescence quenching is characteristic of bimolecular reactions of S₁.^{15,16,17}

Photolysis of riboflavin in its ground singlet state (S_{O}) forms an excited singlet state (S₁). This state can fragment to lumichrome, fluoresce (λ=520 nm, φ_{F}=0.26) or relax to form triplet riboflavin (T₁).⁴ The lifetime of the S₁ state is ≈5 ns, the lifetime of the triplet state in deoxygenated water is 1-100 μs.⁴ The triplet state (³RB*, T₁) is readily detected by laser flash photolysis experiments (λₘₐₓ≈650 nm).⁴ It is formed with a quantum yield of 0.7 in solution. The triplet state can also be detected by its phosphorescence and EPR spectra at 77 K.⁴

Electron rich amino acids (tryptophan, tyrosine, histidine and methionine) and nucleotides (guanosine and adenosine monophosphate) quench the fluorescence of RB.⁴ The S₁ state of riboflavin accepts an electron from the amino acid or nucleotide donor to form a flavin radical anion. Electron transfer proceeds on ultrafast time scales upon excitation of flavin adenine mononucleotide (FAD) and enzyme bound flavins.⁴

Flash photolysis studies demonstrate that phenols and indoles quench the triplet (T₁) state of flavins by sequential electron-proton transfer as shown in Scheme 1 for lumiflavin (LF).⁴ We have posited that this type of photochemistry leads to riboflavin-tryptophan covalent adducts with human serum albumin and occular lens proteins. Hemmerich and Knappe have found that structurally similar adducts are formed upon photolysis of lumiflavin and cyclopentadiene.^{4,18}

We have built a time resolved infrared (TRIR) spectrometer with 50 ns time resolution and 16 cm⁻¹ spectral resolution. Although TRIR spectroscopy is less sensitive and has less time resolution than time resolved UV-vis spectroscopy, TRIR spectroscopy yields much more structural information. We have recently obtained exciting results with this technique, which are consistent with the mechanism outlined in Scheme 1. These results have been published in Martin, C. B.; Tsao, M.-L.; Hadad, C. M.; Platz, M. S. “The Reaction of Triplet Flavin with Indole. A Study using Density Functional Theory and Time Resolved Infrared Spectroscopy” J. Am. Chem. Soc. 2002, 124, 7226-7234, incorporated herein by reference.

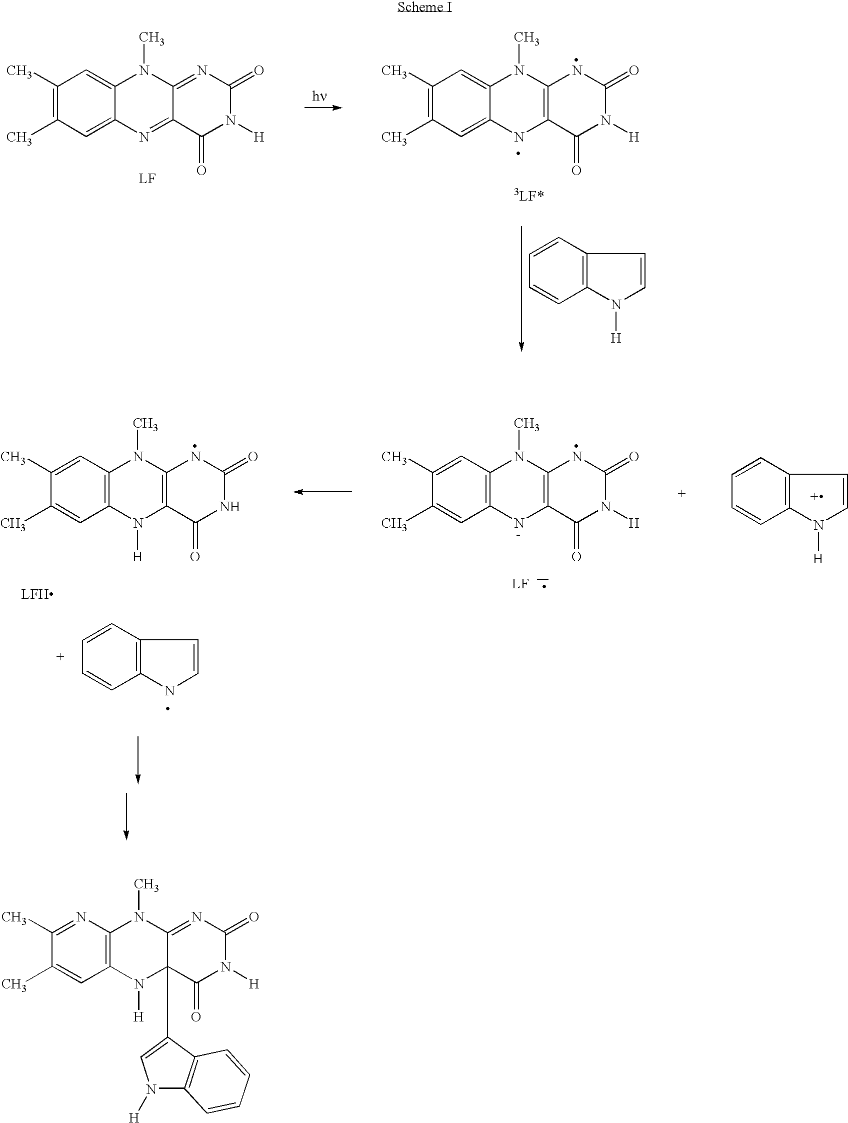

Flash photolysis (355 nm) of riboflavin tetraacetate (RBT) was studied in acetonitrile under nitrogen. In riboflavin tetraacetate the four hydroxyl groups of the ribityl sugar side chain of riboflavin have been acetylated (Scheme II). Riboflavin tetraacetate was studied because of its relatively good solubility in acetonitrile. Acetonitrile is a more convenient solvent than water since it obscures less of the IR spectrum.

In TRIR spectroscopy, regions of positive differential absorption at 1680 cm⁻¹ are due to the formation of a new species, and regions of negative differential absorption are due to the disappearance of riboflavin tetraacetate (1700, 1730 cm⁻¹) in its ground state (S₀). There are regions of zero differential transient absorption (1760-1800 cm⁻¹) where the absorption of photoproduct and riboflavin tetraacetate is the same.

Flash photolysis of RBT produces a transient absorbing at 1660 cm⁻¹ that has a lifetime of ≈2 μs. As it decays riboflavin tetraacetate is reformed with the same time constant, within experimental error. Oxygen shortens the lifetime of the photoproduct to 200 ns. The transient absorption is assigned to triplet riboflavin tetraacetate based on the kinetic behavior of the transient and upon DFT calculations (B3LYP-6-31G*) of triplet lumiflavin. Calculations were performed on lumiflavin and its triplet state because it has a simple methyl group instead of the large ribityltetraacetate side chain. This minimizes the required computational time. We believe that LF is a valid computational model of RB and RBT because the side chain does not conjugate with the π system of the flavin. The calculations predict that the ground state vibrations of the carbonyl groups of LF (and presumably RBT) will shift from 1743.8 and 1736.1 cm⁻¹ to 1682.3 and 1633.2 cm⁻¹ in the triplet state, in excellent agreement with experiment.

Flash photolysis of riboflavin tetraacetate in the presence of 20 mM sodium iodide generates a new transient with maximum absorption at 1636 cm¹ and a lifetime of 16 μs in deoxygenated acetonitrile. This species is assigned to the flavin radical anion (Scheme II). DFT calculations on lumiflavin derived intermediates clearly demonstrate that N⁵ is the site of greatest negative charge of the LF radical anion (e.g., the resonance structure shown below is favored). Thus it will be the position most likely to accept a proton to form a lumiflavin radical (LFH) as shown in Schemes I and II. The computational results should be valid for the larger riboflavin system, as the side chain does not conjugate with the π system of the flavin.

Flash photolysis of RBT in the presence of 20 mM indole or 20 mM phenol produces a transient with absorption maximum at 1664 cm⁻¹. This transient has an absorption maximum similar to the flavin triplet (3RBT*) but lacks the characteristic C═N vibrations of the triplet flavin. The new transient has a much longer lifetime (10 μs) than the flavin triplet and is assigned to neutral radical RBTH (Schemes I and II).

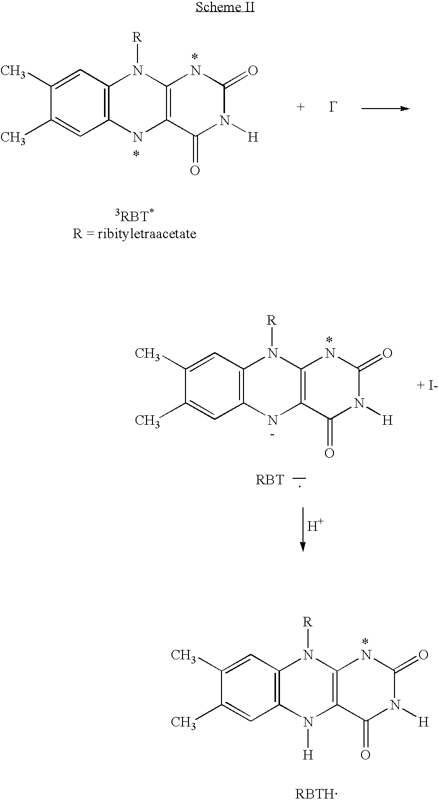

The RBT radical anion can accept a proton at numerous positions to form a series of isomeric neutral radicals. We have used DFT theory to calculate the energies and vibrational spectra of every neutral radical that can be obtained upon protonation of the lumiflavin radical anion. The radical pictured in Schemes I and II is the most stable isomer by 8 kcal/mol. It is also the only low energy flavin radical with a prominent carbonyl vibration predicted (1650 cm⁻¹) to be close to the observed value (1664 cm⁻¹). Thus TRIR spectroscopy combined with DFT calculations allowed us to identify the precise reactive intermediate produced upon photolysis of RBT and indole or phenol in acetonitrile.

We have also applied this methodology to the study of the photochemistry of RBT and adenosine triacetate and Foote's¹⁹ organic soluble di-tert butylmethylsilyl guanosine (G′). The triplet flavin reacts by the same electron-transfer-proton transfer mechanism shown in Scheme I to form the same RBTH radical produced in the flavin triplet reaction with indole. Adenosine triacetate reacts with triplet RBT with a rate constant of 1×10 M⁻¹'s⁻¹ and the silylated guanosine (G′) reacts with a rate constant of 2×10⁸M⁻¹s⁻¹. Ribose tetraacetate does not react with triplet RBT at an appreciable rate (k<1×10⁵M⁻¹s⁻¹)

We have discovered that quantities of G′ sufficient to quench >99% of the fluorescence of RBT lead to the formation of the neutral radical RBTH. Thus reactions of flavin singlet S₁ and triplet T₁ with silylated guanosine both produce the same radical by sequential electron and proton transfer with purine nucleosides.

Other Photosensitizers

In other embodiments the infected EBV transformed lymphocytes are incubated with a flavin-containing photosensitizer other than riboflavin, i.e. an LC resistant flavin photosensitizer. An example of such photosensitizer is shown below:

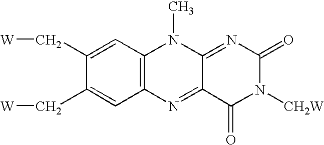

In this structure W is either a hydrogen atom, a simple sugar derivative of ribose or glucose, or a mono, di or tri ethylene glycol. These flavins will be water soluble and electrically neutral. The latter property will allow the flavins to pass though cell membranes. These structures will be much more resistant to photodegradation to lumichrome than riboflavin. This makes these flavins catalytic rather than stoichiometric.

Incorporation of the RB and the LC-Resistant Photosensitizer into the Cells

The EBV-transformed B lymphocytes are incubated in medium containing RB or the LC-resistant photosensitizer under conditions which permit accumulation of RB or the LC-resistant photosensitizer in the transformed lymphocytes and binding of the respective photosensitizer to cellular DNA. The cells which have accumulated RB or the LC-resistant photosensitizer therein are hereinafter collectively referred to as “RB-treated EBV-transformed cells.” Following photoradiation these cells contain metabolic breakdown products of RB or the LC-resistant photosensitizer and are collectively referred to hereinafter as “RB-sensitized EBV-transformed cells.” To enhance binding to cellular DNA, it is preferred that the a substantial portion, i.e., greater than 25%, preferably greater than 35%, more preferably greater than 50% of the EBV-transformed lymphocytes in the cell sample be in S phase. During the S Phase, the cell is actively undergoing DNA synthesis. At the end of the S phase, and just before cell division, it will contain two sets of DNA. It is proposed that cells will be most likely to bind riboflavin and to undergo the desired riboflavin DNA photochemistry at this point of their cycle. Synchronizing the cells present in the sample maximizes the number of cells in S Phase, and thereby minimizes the quantity of riboflavin free in solution, and the risk of toxic cell death, i.e., necrosis.

Synchronization of the cells can be achieved with a drug, such as aphidicolin, which is a DNA polymerase inhibitor. This drug prevents the cells from entering S-phase, the phase during which the cells replicate their DNA. In the presence of aphidicolin those cells already in the posterior phases are killed and the remaining cells accumulate at the beginning of the S phase.

The effects of aphidicolin are reversible. The cells start growing and dividing soon after the drug is removed by washing (during washing the cells are concentrated by centrifugation, the supernatant is discarded and the cells are resuspended in non-treated media.).

The amount of RB incorporated into the medium is an amount sufficient to induce apoptosis of the RB-sensitized EBV-transformed lymphocytes. As taught herein, optimal concentrations may be readily determined by those skilled in the art without undue experimentation. Preferably, the smallest efficacious concentration of RB is used.

Apoptosis is a highly ordered genetically programmed cell death, which results in DNA degradation and nuclear condensation. It is activated by internal signals from the cell itself. In contrast, necrosis is death due to external injury to the cells. Cellular necrosis is a form of cell death that involves a swelling of the cells and membrane rupture. Apoptotic cell death is typically accompanied by one or more characteristic morphological and biochemical changes in cells, such as condensation of cytoplasm, loss of plasma membrane microvilli, segmentation of the nucleus, degradation of chromosomal DNA or loss of mitochondrial function. A recognized biochemical marker of apoptosis is the cleavage of chromatin into nucleosomal fragments. During apoptosis, cells can present signals, which can be used to induce significant immune responses. Levels of apoptosis and necrosis of the RB-sensitized EBV-transformed lymphocytes can be determined via flow cytometry using a technique described by Vermes.³²

In the process of apoptosis, many changes occur in the cell. One of these changes is the translocation of phophatidylserine from the inside face of the plasma membrane to the outside. This change can be detected using a probe which has high affinity to phosphatidylserine called Annexin V. If Annexin V is complexed to a fluorochrome, such as fluorescien isothiocyanate (FITC), it can be detected by a flow cytometer.

The translocation of phosphatidylserine also occurs as a result of necrotic cell death, however. One distinction between apoptotic cell death and necrotic cell death is that during the early stages of apoptosis, the outer membrane of the cell remains intact. After necrosis occurs, the membrane becomes leaky and allows substances to pass through. Consequently, one can use the fluorescent dye DNA stain propidium iodide (PI), which only passes through leaky membranes, as a membrane exclusion dye. In this way, only necrotic cells which allow PI to pass through will be PI positive, while normal and apoptotic cells will be PI negative.

In summary, using Annexin V-PI staining, if cells are normal they will be negative for Annexin V and negative for PI staining. If cells are apoptotic, they will be positive for Annexin V and negative for PI. If cells are necrotic, they will be positive for both Annexin V and PI. This is represented by a flow cytometer as a density plot (note that each point represents a cell, whose X coordinate is a function of how much Annexin V is detected on the cell and whose Y coordinate is a function of how much PI is detected in the cell). Other examples of assays for apoptosis are as follows:

Comet (Single-Cell Gel Electrophoresis) Assay to Detect Damaged DNA

The Comet assay, or single-cell gel electrophoresis assay, is used for rapid detection and quantitation of DNA damage from single cells. The Comet assay is based on the alkaline lysis of labile DNA at sites of damage. Cells are immobilized in a thin agarose matrix on slides and gently lysed. When subjected to electrophoresis, the unwound, relaxed DNA migrates out of the cells. After staining with a nucleic acid stain, cells that have accumulated DNA damage appear as bright fluorescent comets, with tails of DNA fragmentation or unwinding. In contrast, cells with normal, undamaged DNA appear as round dots, because their intact DNA does not migrate out of the cell.

TUNEL Assay

When DNA strands are cleaved or nicked by nucleases, a large number of 3′-hydroxyl ends are exposed. In the TUNEL assay (terminal deoxynucleotidyl transferase dUTP nick end labeling), these ends are labeled with UTP using mammalian terminal deoxynucleotidyl transferase (TdT), which covalently adds labeled nucleotides to the 3′-hydroxyl ends of these DNA fragments in a template-independent fashion. The UTP is then detected using specific probes (e.g., you can incorporate BrdUTP and then use a fluorescent anti-BrdU antibody). The assay can be used on cells in situ or the cells can be analyzed by flow cytometry.

Apoptosis Assays Using Annexin V Conjugates

The human anticoagulant annexin V is a 35-36 kilodalton, Ca²⁺-dependent phospholipid-binding protein that has a high affinity for phosphatidylserine (PS). In normal viable cells, PS is located on the cytoplasmic surface of the cell membrane. However, in apoptotic cells, PS is translocated from the inner to the outer leaflet of the plasma membrane, where it is associated with lipid “rafts”-regions of the plasma membrane that are insoluble in detergents, high in cholesterol and sphingolipids, that sequester glycosylphosphatidylinositol (GPI)-linked proteins and tyrosine-phosphorylated proteins and that seem to be involved in signal transduction. Annexin V that is conjugated to various detectable molecules (i.e., fluorescent molecules) are reacted with cells thought to be undergoing apoptosis. If PS is located on the outer surface of the plasma membrane, the annexin V conjugate will bind and be detectable.

Apoptosis Assays Based on Protease Activity

Members of the caspase (CED-3/ICE) family of proteases are crucial mediators of the complex biochemical events associated with apoptosis. In particular, caspase-3 (CPP32/apopain), which has a substrate specificity for the amino acid sequence Asp-Glu-Val-Asp (DEVD), cleaves a number of different proteins, including poly(ADP-ribose) polymerase (PARP), DNA-dependent protein kinase, protein kinase, and actin. Procaspase-3 is released from the mitochondria into the cytoplasm during apoptosis and activated to caspase-3 by an as-yet-unknown enzyme. Assays for caspase comprise addition of substrates for the enzyme that, for example, increase their fluorescence upon cleavage by caspase-3.

Addition of a Non-Toxic Antioxidant

It is well known that long-lived oxidants such as hydrogen peroxide and superoxide ion are produced when riboflavin in water or growth medium is exposed to visible light.²⁰ These effects are enhanced in the presence of electron donors such as tryptophan and tyrosine. Short lived oxidants such as singlet oxygen are also formed upon photolysis of riboflavin.⁴

The first step in the formation of long-lived oxidants involves electron transfer from a donor (tryptophan, tyrosine, ground state flavin) to either the excited singlet or triplet state of riboflavin to form the flavin radical anion (RB⁻).⁴

There are many plausible mechanisms by which reactive oxygen species (ROS) can be formed from the radical anion of the flavin. An electron can be transferred from the radical anion of riboflavin to oxygen to form superoxide ion (Scheme III).

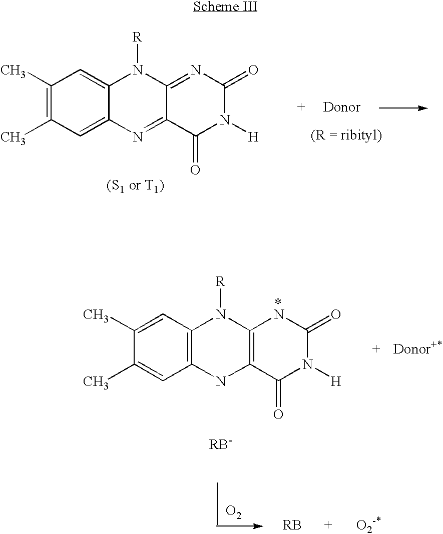

Alternatively, protonation of the reduced flavin forms a neutral radical RBH. which can react with oxygen to form a hydroperoxy radical (RBOO., Scheme IV).⁴

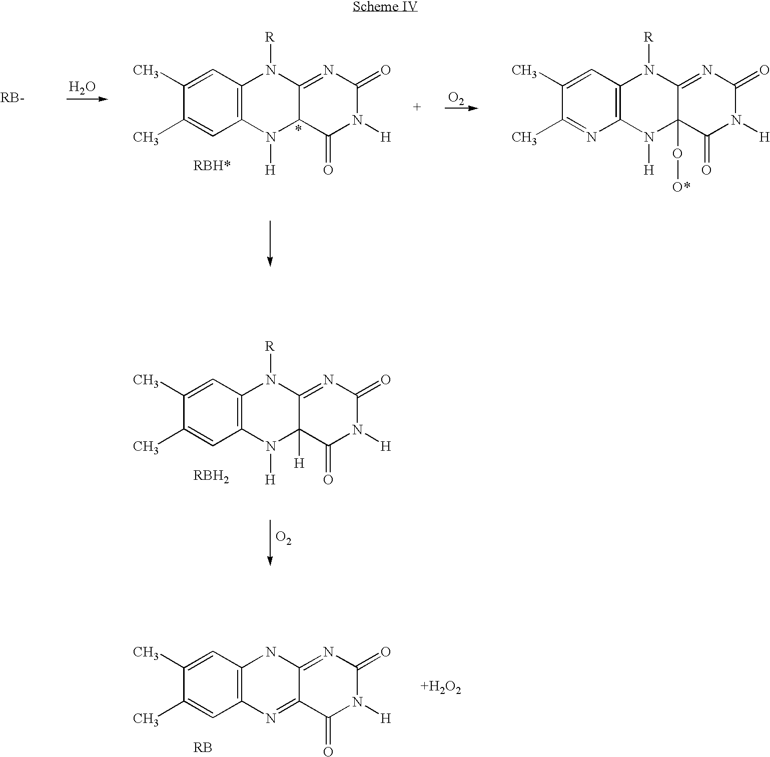

Complete reduction of riboflavin forms leukoflavin, RBH₂.^{4,21} This species (RBH₂, Scheme IV) is stable in the absence of oxygen. However, it reacts very rapidly with oxygen to form hydrogen peroxide and to regenerate riboflavin. In this manner the photochemical formation of hydrogen peroxide can be catalytic rather than stoichiometric in riboflavin. The simplicity of the overall reaction of leukoflavin with oxygen is deceptive. The overall reaction is actually a multistep sequence involving superoxide ion.^{4,21}

Hydrogen peroxide and superoxide ion both react with guanosine residues of cellular DNA. Isolation and digestion of the cellular DNA yields either 8-oxoguanosine or 8-oxoguanine (shown below) depending on the methodology employed to digest the nucleic acid.²² Hydrogen peroxide also induces single strand breaks when added to nucleic acids,²³ and is toxic to cells, of course.²⁴

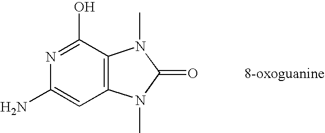

Riboflavin sensitized photolysis of DNA produces 8-oxoguanosine and single strand breaks²⁵ in addition to adducts of flavin and nucleic acid.²⁶ Cadet and co-workers²⁷ have shown that excited riboflavin can accept an electron from guanosine to form the guanosine radical cation. Subsequent hydrolysis of the guanosine radical cation also produces 8-oxoguanosine along with other products. Thus 8-oxoguanosine can be formed by more than one mechanism and at least by one mechanism without the intervention of singlet oxygen, hydrogen peroxide or superoxide ion.

Yamamoto, Nishimura and Kasai have analyzed the DNA of cultured mammalian cells (mouse lymphoma line L5178Y) exposed to riboflavin and visible light and found the formation of 8-oxoguanosine.²⁸ Previously Hoffman and Meneghini discovered that photolysis of green monkey kidney cells and riboflavin led to the formation of single strand breaks of the cellular DNA.²⁹ Thus, it is a straightforward conclusion that photolysis of intracellular riboflavin produces intracellular oxidants that damage cellular DNA and/or that photolysis of extracellular riboflavin generates extracellular hydrogen peroxide that can passively transport to the cell nucleus and damage the cellular DNA. There seems little doubt that this is the mechanism for much cellular DNA damage but Cadet's work²⁴ indicates that it is probably not the only intracellular riboflavin sensitized photochemistry that transpires. Furthermore, Ennever and Speck²⁶ have demonstrated the flavin-nucleic acid adducts are formed when riboflavin and nucleic acids are exposed to visible light. The formation of these adducts is not oxygen dependent. The structure of these adducts, discovered in 1983, remains unknown.

To reduce or prevent nonspecific cellular damage, i.e., damage to the cell membrane rather than the DNA, of the EBV-transformed B lymphocytes from photolysed breakdown products of RB or the LC-resistant photosensitizer, and thereby favor cellular apoptosis as opposed to cellular necrosis, a non-toxic antioxidant is added to the medium of the EBV-transformed B lymphocytes. As used herein the term “non-toxic antioxidant” refers to a compound, preferably a physiological compound, that, at concentrations which are non-toxic to cells, is capable of reducing or inhibiting formation of the long-lived oxidants that are formed when RB or the LC-resistant flavin photosensitizer is exposed to visible light. “Non-toxic to cells” means that cell death or prevention of cell growth are minimized or avoided altogether. The non-toxic antioxidant is added to the medium prior to photoradiation of the RB-treated EBV-transformed B lymphocytes. Thus, the non-toxic antioxidant may be added concurrently with RB or the LC-resistant flavin photosensitizer or following accumulation, i.e., equilibration, of RB or the LC-resistant flavin photosensitizer in the cell. Glutathione is one antioxidant that has been found particularly useful for the present application.

The amount of non-toxic antioxidant used depends on the concentration of the photosensitizer, and amount of light exposure. As taught herein, optimal concentrations of the non-toxic antioxidant may be readily determined by those skilled in the art without undue experimentation. Preferably, the smallest efficacious concentration of the non-toxic antioxidant is used.

Photoradiation of the RB-treated EBV-Transformed Lymphocytes

The RB-treated EBV-transformed B lymphocytes are exposed to photoradiation of the appropriate wavelength to activate RB or the LC-resistant flavin photosensitizer, using an amount of photoradiation sufficient to activate RB or the LC-resistant flavin photosensitizer, but less than that which would cause substantial non-photosensitizer sensitized damage to the biological components of the cell. The wavelength of light used and the amount of radiation used is readily determinable without undue experimentation by one of ordinary skill in the art, using literature sources or direct undue experimentation by one of ordinary skill in the art, using literature sources or direct measurement. Preferably the light source is a visible light source providing 400 nm to about 700 nm, and more preferably about 400 nm to about 500 nm of radiation. All other parameters that may be involved in preparing a cell preparation comprising a plurality of apoptotic EBV-transformed lymphocytes, including appropriate temperatures for the incubation and photoradiation steps as well as the ranges of temperature, photoradiation intensity and duration, and photosensitizer and non-toxic antioxidant concentrations which will optimize apoptosis and minimize damage to EBV proteins and the cellular membrane also easily determined as is known in the art or readily determinable without undue experimentation by one of ordinary skill in the art, using literature sources or direct measurement.

Purification of the Cell Preparation

In addition to the apoptotic EBV-transformed lymphocytes, the cell preparation further comprises metabolic breakdown products of riboflavin or the LC-resistant flavin photosensitizer. If desired, these breakdown products can be substantially removed from the preparation using standard techniques to provide a partially purified preparation of in activated EBV-transformed cells. For example, the photosensitized cell preparation may be subjected to low speed centrifugation to pellet the cells. The supernatant which contains the extracellular metabolic breakdown products is discarded and the cells collected. Additional washing steps with fresh medium may be used to remove residual extracellular materials and to further purify the cell preparation.

Prematurely born infants often have immature livers that can not degrade bilirubin (BR), a metabolite of hemoglobin, to smaller, more water soluble compounds which can then be excreted.⁸ These jaundiced infants are commonly treated by exposure to visible light (447 nm). BR in superficial tissues of neonates absorbs the radiation and forms excited triplet states. The bilirubin triplet state sensitizes the formation of singlet oxygen, and the singlet oxygen so formed attacks and degrades ground state BR to smaller, more water soluble molecules. RB circulating in the blood of neonates strongly absorbs visible light and is also excited when neonates are treated for hyperbilirubinaemia. Consequently, the blood level of RB in neonates treated with phototherapy is dramatically depleted.⁸ The fate of the missing RB is not known with certainty, but some RB is likely converted to LC and to albumin adducts in the neonates.

There is a hereditary trait common, but not limited to people of Amish descent known as Crigler-Najjar Syndrome.⁹ These individuals cannot degrade BR and undergo BR (and inadvertently riboflavin) phototherapy throughout their lives, or until liver transplantation is possible. No unusual health effects have been observed as a consequence of long term phototherapy, the photolysis of riboflavin in their blood and their subsequent long-term exposure to RB breakdown products. One individual has received whole body phototherapy from birth for over ten years without apparent, unusual health concerns.¹⁰

Studies in Denmark have considered the possibility that bilirubin (and inadvertently riboflavin) phototherapy might promote cancer.¹¹ Consequently, they followed over 50,000 neonates receiving BR phototherapy for decades. The number of cancers predicted for this cohort was 85. A total of 87 cancers were found upon cross checking this group against the national cancer registry of Denmark, a result which was considered statistically insignificant.¹¹ In contrast, it has been concluded that psoralen and UVA phototherapy (PUVA) of psoriasis and subcutaneous lymphoma leads to statistical increases in squamous cell carcinoma (SSC).¹² Extensive experience with RB phototherapy under conditions relevant to blood banking indicates that RB photolysis in vivo does not lead to increased incidence of cancer. In conclusion, these studies indicate that photolysis-induced breakdown products of RB are not harmful to human subjects. Thus, it is expected that the cell preparations prepared as described above may be used without further purification.

Uses of the Cell Preparations

The “non-purified” and partially-purified cell preparations may be used to elicit an immune response either in vitro or in vivo and to treat human subjects, particularly children, who are candidates for an organ transplant. Preferably, the cell preparations that are used to treat organ transplant candidates comprise autologous apoptotic EBV-transformed lymphocytes.

Eliciting an Immune Response in Vitro

The cell preparation or the isolated cells contained within the preparation are contacted with peripheral blood under conditions which permit activation and/or proliferation of T lymphocytes.

Eliciting an Immune Response In Vivo

A therapeutically effective amount of the cell preparation or the partially purified preparation of RB sensitized EBV-transformed B cells is administered to a human subject, preferably to subject who has little to no circulating levels of EBV-specific T cells. The cell preparation is said to be administered in a “therapeutically effective amount” if the amount administered is physiologically significant. An agent is physiologically significant if its presence results in a detectable change in the physiology of a recipient mammal. In particular, a cell preparation of the present invention is physiologically significant if its presence invokes a cellular immune response in the recipient mammal. This amount is determined using standard techniques. Preferably, this amount is determined by measuring the levels of circulating T cells specific against EBV.

The partially purified or non-purified cell preparation can be combined in admixture with an pharmaceutically acceptable carrier or diluent. Optionally, the partially purified or non-purified cell preparation can be prepared in admixture with an adjuvant. The term “adjuvant” as used herein refers to a compound or mixture which enhances the immune response to an antigen. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Selection of an adjuvant depends of the animal subject to be vaccinated. Preferably, a pharmaceutically acceptable adjuvant is used. For example, oils or hydrocarbon emulsion adjuvants should not be used for human. One example of an adjuvant suitable for use with humans is alum (alumina gel.)

Preferably, the cell-based immunogenic compositions are administered to the human subject by injection, such as for example intramuscular (i.m.), intradermal (i.d.), intranasal (i.n.) or sub-cutaneous (s.c.) injection. It is contemplated that two or more injections over an extended period of time will be optimal. Preferably, the immunogenic compositions are administered in an dosage sufficient to prevent, reduce or retard development of PTLD in a subject through a series of immunization challenge studies using a suitable animal host system, e.g. transgenic mice which are thought to be an acceptable standard for human use considerations.

The dosage to be administered depends on the size of the subject being treated as well as the frequency of administration and route of administration. Ultimately, the dosage will be determined using clinical trials. Initially, the clinician will administer doses that have been derived from animal studies.

Preferably, the cell preparation is administered to the subject prior to organ transplantation and initiation of immunosuppressive medications.

### EXAMPLES

The following examples are for purposes of illustration only and are not intended to limit the scope of the claims which are appended hereto.

### Example 1

### Riboflavin Binding Affinities

Riboflavin is known to bind to nucleic acid. Using Scatchard plots, Kuratomi and Kobayashi have found that one riboflavin is bound to every 500 nucleotides of native DNA.¹³ We surveyed and compared the binding affinities of riboflavin and two other common sensitizers, AMT and methylene blue, for cellular and plasma components using a simple two chamber dialysis assay. In chamber 1 is placed a macromolecule such as bovine albumin, calf thymus DNA, phosphatidylserine (which has a net negative charge) or phosphatidylcholine, which has no net charge. The sensitizer is placed in the second chamber and its absorbance is recorded. The two chambers are separated by a semi-permeable membrane with a molecular weight cut of 3500 amu, which permits diffusion of only the sensitizer. If there is no binding of sensitizer to macromolecule the absorbance of chamber 2 will drop by 50% at equilibrium. Binding is demonstrated by a decrease in the absorbance of the sensitizer in chamber 2 of greater than 50%.

The results are given in Table 1¹⁴ which reveal that 7% of the riboflavin is bound to calf thymus DNA under these conditions. There is no detectable association of riboflavin with bovine albumin or either phospholipid. Methylene blue which is positively charged associates strongly with calf thymus DNA and with the negatively charged phospholipid vesicle (phosphatidyl serine) but has little affinity for either bovine albumin or phosphatidylcholine. AMT, a positively charged psoralen, has very high affinity for all of the macromolecules (DNA, protein, lipid) assayed. The absolute affinity of riboflavin for DNA lags behind that of methylene blue and AMT. The modest absolute affinity of riboflavin for DNA explains its lack of mutagenicity and its superior safety relative to the other sensitizers. Furthermore, it is not the absolute affinity of the sensitizer for DNA that is most important. Rather it is the ability of the sensitizer to accumulate only in the nucleic acid of the cell or the virus, in the presence of plasma protein and phospholipid cell membranes, that is most important.

TABLE 1
Percent of sensitizer bound to macromolecule
after 24 hours of dialysis (average of n = 3).¹⁴

| | | | Neutral | Negatively Charged |
|---|---|---|---|---|
| | Albumin^{a} | DNA^{b} | Lipid^{c} | Lipid^{d} |
| AMT | 17% | 30% | 16% | 53% |
| MB | <3% | 65% | <3% | 20% |
| RB | <3% | 7% | <3% | <3% |

a)bovine albumin, 75 mg/mL; [sensitizer] ≈1.1 × 10⁻³ M
b)calf thymus DNA, [nucleotides] = 1.0 × 10⁻³ M, [AMT] = 1.09 × 10⁻³ M, [DMMB] = 7.7 × 10⁻⁴ M, [MB] = 6 × 10⁻⁴ M, [RB] = 3.9 × 10⁻⁴ M
c)L-α-phosphatidylcholine, 4.17 mg/mL; [RB] = 1.3 × 10⁻³ M; [MB] = 1.0 × 10⁻³ M; [DMMB] = 1.5 × 10⁻³ M; [AMT] = 1.5 × 10⁻³ M
d)1,2-Dimyristoyl-sn-glycero-3-phosphatidylserine sodium salt, 4.17 mg/mL; [RB] = 1.11 × 10⁻³ M; [MB] 1.03 × 10⁻³ M; [AMT] = 1.32 × 10⁻³ M

In this application human apoptosis and inactivation of EBV-transformed B lymphocytes by selective sensitization of damage to the cellular nucleic acid. Thus, it is highly preferred that the sensitizer recognize chemical differences between the different components of the cell. Of the three sensitizers surveyed, riboflavin is the sensitizer most likely to associate selectively in the nucleic acid of the cell.

Riboflavin, methylene blue and AMT are all water soluble. Methylene blue and AMT achieve water solubility by the presence of a positive charge. Riboflavin is fundamentally different as it is electrically neutral. Because riboflavin is uncharged it has the greater likelihood of passive transport through membranes and reaching the nucleic acid target of the pathogen.

### Example 2

### Riboflavin Sensitized Killing of Human Lymphoblastoid Cells Infected with the Epstein Barr Virus

Photolysis (436 nm) of riboflavin (25-100 μL) in aerated RPMI growth medium produces hydrogen peroxide. The yield of hydrogen peroxide correlates with the concentration of riboflavin and the length of exposure of the sample to light. (Table 2).

TABLE 2
The yield of hydrogen peroxide formed upon exposure of
solutions of riboflavin in commercial RPMI growth medium,
to visible light, as a function of exposure time. The peroxide
yield was measured with starch-iodide paper.

| Time (minutes) | Media + 0.1 mM RB |
|---|---|
| 0 | 1 mg/L H₂O₂ |
| 20 | 12 mg/L H₂O₂ |
| 40 | 20 mg/L H₂O₂ |
| 60 | 30 mg/L H₂O₂ |

EBV infected human lymphoblastoid cells (LCLs) were added to commercial solutions of RPMI growth medium that had been previously photolyzed in the presence of riboflavin. After 24 hours of incubation, 100% of the cells were dead as discerned by cell counts using a hemacytometer with Trypan Blue staining. However, photolysis (436 nm) of an RPMI solution that did not contain riboflavin did not result in any increased levels of dead cells.

We have found that these cells grow normally in the presence of 10 mM glutathione, a physiological antioxidant. Solutions of riboflavin (100 μM) and glutathione (10 mM) were photolyzed. Cells (0.50×106 cells/mL) were added to these photolyzed solutions and incubated and counted after 24 hours. After 24 hours of incubation, the number of living cells had grown to 0.65×106 cells/mL. The control group of cells (0.50×106 cells/mL) grew to 0.75×106 cells/mL upon incubation in unphotolyzed RPMI growth medium. Clearly, a significant number of cells survived exposure to the previously photolyzed solution of riboflavin and glutathione and continued to reproduce. Thus, glutathione neutralizes the long-lived oxidants produced on photolysis of riboflavin.

LCLs were then photolyzed in the presence of both 100 μM riboflavin and 10 mM glutathione, incubated and counted after 24 hours. Upon photolysis of cells in the presence of riboflavin and glutathione, 72% of the recovered cells were dead, as opposed to 100% death seen with RB alone. Thus, as we have established that glutathione neutralizes long-lived oxidants, it appears likely, that photolysis of DNA bound riboflavin was responsible for cell death.

In summary, we have shown that there are essentially two distinct mechanisms of cell death during photolysis of RB. The first is a nonspecific damage from photolysed breakdown products from RB which can be neutralized by the addition of an antioxidant such as glutathione. This is represented in our system by photolysis of solutions before the addition of cells. The second is a specific killing associated with damage due to photolysis of RB directly complexed to human LCLs and quite possibly to their cellular DNA, which would be less susceptible to inhibition by the addition of an antioxidant. We study this method of killing in our system with phololysis of solutions in the presence of the cells. In this way, we are able to use glutathione to focus on the second more specific form of cell killing.

### Example 3

### Optimization of the Photolysis

Glutathione Efficiency.

This experiment has been performed to see if the amount of glutathione could be reduced without reducing the efficiency of the protection against the ROS.

Seven 5-mL solutions of media+100 μM RB containing different concentrations of glutathione were photolyzed for 2 hours, then added to 3.5×10⁶ healthy cells (0.7×10⁶ cells/mL) and put in the incubator for 24 hours.

Solutions: #1=media alone

#2=media+100 μM RB

#3=media+100 μM RB+0.1 mM of glutathione

#4=media+100 M RB+0.5 mM of glutathione

#5=media+100 μM RB+1 mM of glutathione

#6=media+100 μM RB+5 mM of glutathione

#7=media+100 μM RB+10 mM of glutathione

TABLE 3
Cells count 24 hours after the photolysis of the solutions

| Flask | Dead/ml | Alive/mL | % Dead |
|---|---|---|---|
| #1 | 0 | 1.48 × 10⁶ | 0 |
| #2 | 0.93 × 10⁶ | 0.2 × 10⁶ | 82 |
| #3 | 0.6 × 10⁶ | 0.1 × 10⁶ | 85.7 |
| #4 | 0.7 × 10⁶ | 0.15 × 10⁶ | 82.4 |
| #5 | 0.6 × 10⁶ | 0.15 × 10⁶ | 80 |
| #6 | 0.42 × 10⁶ | 0.25 × 10⁶ | 62.7 |
| #7 | 0.3 × 10⁶ | 0.78 × 10⁶ | 28 |

5 mM of glutathione reduces the number of cells killed but does not provide an efficient protection.

By reducing the concentration of riboflavin used without altering the percentage of cells killed, it would be possible to decrease the quantity of glutathione as well.

Riboflavin Efficiency.

5 different concentrations of riboflavin (and one negative control) in media were tested in their ability to kill the cells upon photolysis.

Solutions: #1 contains no RB

#2 contains 10 μM RB

#3 contains 25 μM RB

#4 contains 50 μM RB

#5 contains 75 μM RB

#6 contains 100 μM RB

3.5×10⁶ cells were put into each well (0.7×10⁶ cells/mL, 5 mL of solution) and allowed to equilibrate for 24 hours in the incubator. The glutathione will be added just before the photolysis. Then, the cells are photolyzed for 2 hours, under the 6 bulbs, and they are left in the incubator overnight.

TABLE 4
Cells count after 24 hours in the incubator:

| Flask | Dead/mL | Alive/mL | % Dead |
|---|---|---|---|
| #1 | 0 | 0.88 × 10⁶ | 0 |
| #2 | 0.46 × 10⁶ | 0.58 × 10⁶ | 44 |
| #3 | 0.64 × 10⁶ | 0.41 × 10⁶ | 61 |
| #4 | 0.49 × 10⁶ | 0.56 × 10⁶ | 47 |
| #5 | 0.53 × 10⁶ | 0.44 × 10⁶ | 55 |
| #6 | 0.64 × 10⁶ | 0.31 × 10⁶ | 67 |

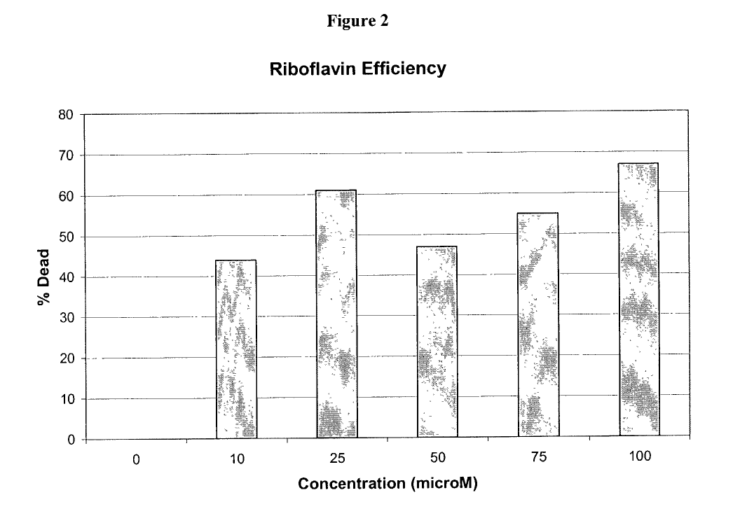

As shown in FIG. 2, 25 μM of riboflavin killing roughly as many cells as 75 or 100 μM, it looks like we can reduce the concentration of riboflavin used to kill the cells.

Riboflavin Equilibration Time.

The equilibration time used so far (24 hours in the incubator) being very long, it seemed possible to reduce it without altering the efficiency of the incorporation of the riboflavin into the DNA.

4 time points have been tested: 1 h, 2 h, 3 h, and 4 h of equilibration of the cells in a solution containing the riboflavin. The experiment was done with 3 different concentrations of RB (0 μM as a control, 100 and 50 μM) since we saw that we could reduce it. Thus, 12 flasks containing 7 millions cells in 10 mL of media have been prepared.

Before photolysis and after equilibration the cells are counted:

TABLE 5
Before photolysis and after equilibration the cells are counted:

| | 1 h equilib. | 2 h equilib. | 3 h equilib. | 4 h equilib. |
|---|---|---|---|---|
| 0 μM | 0.41 × 10⁶ c/mL | 0.50 × 10⁶ c/mL | 0.43 × 10⁶ c/mL | 0.45 × 10⁶ c/mL |
| 50 μM | 0.53 × 10⁶ c/mL | 0.50 × 10⁶ c/mL | 0.46 × 10⁶ c/mL | 0.53 × 10⁶ c/mL |
| 100 μM | 0.64 × 10⁶ c/mL | 0.60 × 10⁶ c/mL | 0.52 × 10⁶ c/mL | 0.66 × 10⁶ c/mL |

10 mM of glutathione is added to each flask and the cells were then photolyzed for 2 hours under the 6 bulbs (163.57 lux). They were then left in the incubator for 24 h.

TABLE 6
Percentage of dead cells after 24 h in the incubator:

| | 1 h equilib. | 2 h equilib. | 3 h equilib. | 4 h equilib. |
|---|---|---|---|---|
| 0 μM | 15.9% | 16.5% | 14% | 14.6% |
| 50 μM | 74% | 60.2% | 58% | 49% |
| 100 μM | 64% | 61% | 35% | 37% |

These data suggest that 1 and 2 hours of equilibration is more effective than 3 or 4 h. There is almost no difference in efficiency between the two concentrations used and the percentages of killed cells are close to those obtained with a 24 h equilibration time. With these results, it was possible to investigate further on the amount of riboflavin used.

RB Efficiency/Equilibration Time

4 different concentrations of riboflavin (0, 10, 25, 50 μM) and 2 equilibration time points were tested.

TABLE 7
Before photolysis and after equilibration the cells are counted:

| | 0 μm | 10 μm | 25 μM | 50 μM |
|---|---|---|---|---|
| 1 hour | 1.18 × 10⁶ c/mL | 1.14 × 10⁶ c/mL | 0.58 × 10⁶ c/mL | 0.71 × 10⁶ c/mL |
| equilib. | | | | |
| 2 hours | 0.73 × 10⁶ c/mL | 0.44 × 10⁶ c/mL | 0.37 × 10⁶ c/mL | 0.35 × 10⁶ c/mL |
| equilib. | | | | |

10 mM of glutathione is added to each flask and the cells are photolized for 2 hours. Then they are left in the incubator overnight.

TABLE 8
Percentage of dead cells:

| | 0 μm | 10 μM | 25 μM | 50 μM |
|---|---|---|---|---|
| 1 hour equilib. | 15% | 57% | 56% | 65% |
| 2 hours equilib. | 23% | 69% | 65% | 62% |

Thus, we can reach an acceptable percentage of killings by leaving the cells to equilibrate in the riboflavin for only 2 hours. This allows reducing considerably the length of the experiment.

Concerning the concentration used, we can see that 10 μM gives the same results as 25 or 50 μM; therefore, this concentration was kept for further work.

Photolysis Time

This work was performed at the same time as the other experiments, so the concentration tested was still 50 μM and the equilibration time 24 h. So far, the cells treated with riboflavin were photolized for 2 hours and the point of this experiment was to see if this time could be reduced.

Six flasks containing 7×10⁶ cells in 10 mL of RB-RPMI solution were photolyzed for different times:

#1=50 gM RB; 30 minutes

#2=50 gM RB; 60 minutes

#3=100 gM RB; 60 minutes

#4=100 gM RB; 0 minutes

#5=0 gM RB (RPMI only); 0 minutes

#6=0 gM RB (RPMI only); 60 minutes

#4, 5 and 6 were used as controls.

The cells were put in the incubator overnight to equilibrate in the riboflavin.

Then, they were counted just before photolysis and 10 mM of glutathione was added to each flask; they were photolyzed under air, using the 6 bulbs for the time mentioned above. #4 and 5 were wrapped in aluminum foil and kept outside the incubator for 60 minutes, so that they were standing in the same conditions of temperature and CO₂ concentration as the photolyzed flasks.

TABLE 9
Before photolysis:

| #1 | #2 | #3 | #4 | #5 | #6 |
|---|---|---|---|---|---|
| 0.99 × 10⁶ | 1.11 × 10⁶ | 1.11 × 10⁶ | 1.10 × 10⁶ | 0.82 × 10⁶ | 0.92 × 10⁶ |
| cells/mL | cells/mL | cells/mL | cells/mL | cells/mL | cells/mL |

After photolysis, the cells were put back in the incubator and were counted at different time points (0, 1 h, 3 h, 4 h, 5 h, and 6 h after) to determine the moment they start dying. No significant amount of dead cells was detected at any of these time points; a night in the incubator is necessary to get the expected percentage of dead cells.

TABLE 10
Results after 24 hours:

| Flask | Dead/ml | Alive/mL | % Dead |
|---|---|---|---|
| #1 | 0.58 × 10⁶ | 0.45 × 10⁶ | 56.3 |
| #2 | 0.69 × 10⁶ | 0.39 × 10⁶ | 64 |
| #3 | 0.77 × 10⁶ | 0.49 × 10⁶ | 61 |
| #4 | 0.26 × 10⁶ | 1.15 × 10⁶ | 18.4 |
| #5 | 0.35 × 10⁶ | 1.65 × 10⁶ | 17.5 |
| #6 | 0.31 × 10⁶ | 1.12 × 10⁶ | 21.7 |

The time of photolysis can then be reduced to 1 hour or 30 minutes (considering that #1 and 2 have similar results).

We have systematically varied the time of incubation of cells with riboflavin, the concentrations of riboflavin and glutathione, and the length of photolysis. By doing so, we have optimized our system to maximize the amount of specific killing due to riboflavin-nucleic acid photochemistry while minimizing the amount of nonspecific killing due to long-lived reactive oxygen species. The optimal conditions with LCLs cells infected with Epstein-Barr Virus are 60 minutes of incubation time of the cells with 25 μM riboflavin, the presence of 10 mM glutathione and 60 minutes of exposure to visible light (125 lux).

### Example 4

### Cell Synchronization

To maximize cell death by apoptosis, we wish to maximize the amount of riboflavin bound to cellular DNA.

During the S Phase, the cell is actively undergoing DNA synthesis. At the end of the S Phase, and just before cell division, it will contain two sets of DNA. It is proposed that cells will be most likely to bind riboflavin and to undergo the desired riboflavin DNA photochemistry at this point of their cycle. Synchronizing the cells present in the sample will maximize the number of cells in S Phase, and so minimize the quantity of riboflavin free in solution, that is, the risk of toxic death for the cells.

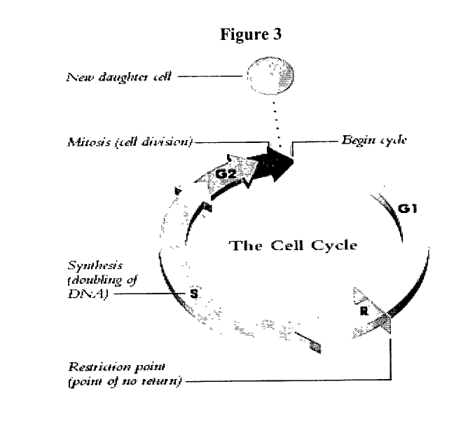

Normally, cells proceed through four stages, known as the cell cycle. This process is schematically described in FIG. 3.

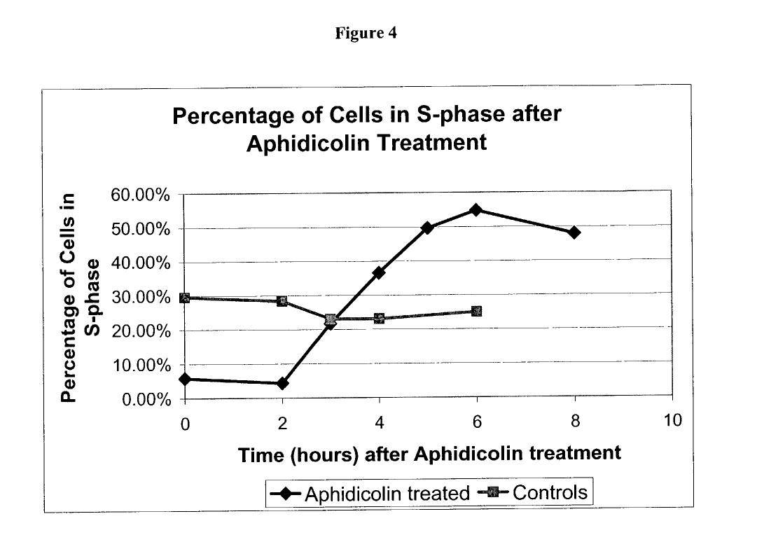

Using a method described by Matherly³⁰ cell synchronization can be achieved with the drug aphidicolin, a DNA polymerase inhibitor. This drug prevents the cells from entering S-phase, the phase during which the cells replicate their DNA. Therefore, the cells accumulate at G1 phase, immediately prior to the start of S phase. These effects of aphidicolin are reversible; the cells return to normal cell-cycling soon after the drug is removed from the culture by washing. Thus, because all of the cells were synchronized together in G1, after removal of aphidicolin, they will proceed through S-phase together. Using flow cytometric analyis of propidium-iodine stained cells, as described by Krishan,³¹ we were able to determine the stage of cell cycle of the cultures, as shown in FIG. 4.

We confirm that aphidicolin treatment synchronizes the cell cycle of the LCLs and significantly increases the proportion of cells in S-phase, with a peak at 6 hours after removal of aphidicolin from solution. Consequently, we hypothesize that treatment with aphidicolin will increase the effectiveness of specific killing via RB-DNA photochemistry.

### Example 5

### Apoptosis Assay

Apoptosis is a highly ordered genetically programmed cell death, which results in DNA degradation and nuclear condensation. It is activated by internal signals from the cell itself. In contrast, necrosis is death due to external injury to the cells. During apoptosis, cells can present signals, which can be used to induce significant immune responses. By taking advantage of this, one can potentially make vaccines by inducing apoptosis in cells. In order to more specifically describe the nature of cell death via RB-DNA photochemistry, we use a technique described by Vermes³² to detect the levels of apoptosis and necrosis of the cells via flow cytometry.

In the process of apoptosis, many changes occur in the cell. One of these changes is the translocation of phophatidylserine from the inside face of the plasma membrane to the outside. This change can be detected using a probe which has high affinity to phosphatidylserine called Annexin V. If Annexin V is complexed to a flurochrome, such as fluorescien isothiocyanate (FITC), it can be detected by a flow cytometer.

The translocation of phosphatidylserine also occurs as a result of necrotic cell death, however. One distinction between apoptotic cell death and necrotic cell death is that during the early stages of apoptosis, the outer membrane of the cell remains intact. After necrosis occurs, the membrane becomes leaky and allows substances to pass through. Consequently, one can use the fluorescent dye DNA stain propidium iodide (PI), which only passes through leaky membranes, as a membrane exclusion dye. In this way, only necrotic cells which allow PI to pass through will be PI positive, while normal and apoptotic cells will be PI negative.

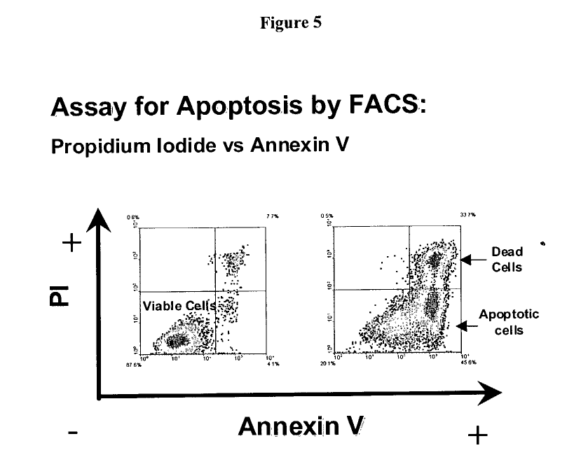

In summary, using Annexin V-PI staining, if cells are normal they will be negative for Annexin V and negative for PI staining. If cells are apoptotic, they will be positive for Annexin V and negative for PI. If cells are necrotic, they will be positive for both Annexin V and PI. This is represented by a flow cytometer as a density plot (note that each point represents a cell, whose X coordinate is a function of how much Annexin V is detected on the cell and whose Y coordinate is a function of how much PI is detected in the cell), as shown in FIG. 5.

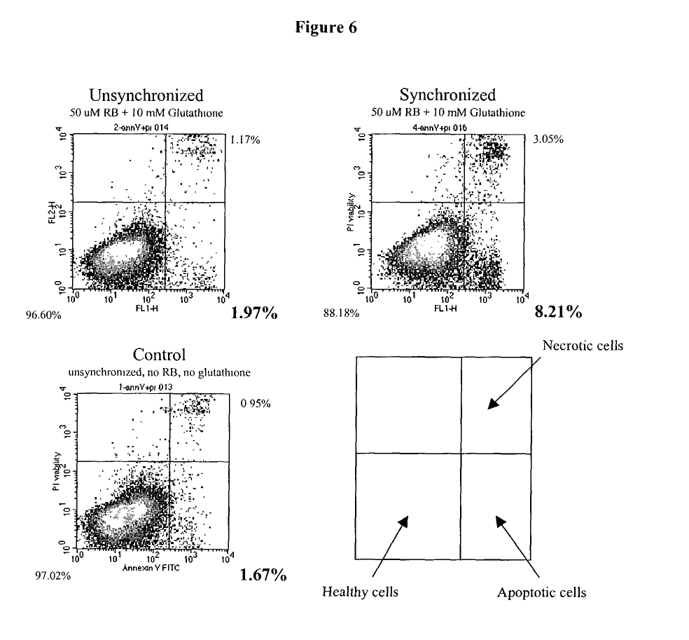

Using this assay we compared synchronized and unsynchronized LCLs in their susceptibility to RB-DNA photolysis. We treated LCLs with aphidicolin for 24 hours and then removed it from culture. At this point, RB was incubated with both the synchronized LCLs and unsynchronized controls for 6 hours, coinciding with maximal levels of synchronized cells in S-phase. We then photolyzed the cells for 1 hour. 8 hours after photolysis, we analyzed the cells using Annexin-PI staining for apoptosis yielding the results shown in FIG. 6.

Thus, we are able to conclude that cell synchronization with aphidicolin increases the effects of RB-DNA photolysis in comparison to the unsynchronized cells. Notably, we see that RB-DNA photolysis is inducing the programmed process of apoptosis in the LCLs (from 1.67% to 8.21%). That we are able to induce apoptosis via RB-DNA photolysis means that the present technique can be used to produce vaccines to provoke immune responses.

### Example 6

### Generation of EBV-Specific Immune Response In Vitro Using RB Treated and Photolyzed Human LCLs

We disclose that RB treated and photolyzed human LCLs can elicit an EBV-specific immune response in vitro when exposed to normal peripheral blood cells from a matching donor. Such an immune response can be created in vitro when EBV+ LCLs are irradiated and exposed to peripheral blood cells from the same donor. In our lab, we have a set of normal, healthy donors, for whom we have a readily available source of peripheral blood and an existing set of in vitro generated EBV+ transformed LCLs.

EBV-transformed LCLs obtained from several donors are treated with RB and subjected to photoradiation. The resulting RB-sensitized EBV-transfromed LCLs are then contacted with peripheral blood cells obtained from the respective matching donor. In the immune response generated in this co-culture, we expect to see a proliferation of EBV-specific Cytotoxic T Lymphocytes. These T cells are thought to expand from a population of existing EBV-specific memory T cells exist that respond by proliferating and gaining anti-tumor activity.³³ This proliferation can be measured by quantitating the absolute number of cells generated in culture and analyzing these populations using flow cytometry to identify CD3+ CD8+ CD44+ T cells.³⁴ These antigens identify T cells [CD3], Cytotoxic T cell subclass [CD8], and memory phenotype [CD44]. In addition to these antigens, we have molecular markers called MHC Class I tetramers that are specific for human MHC haplotypes that can identify T cells that are specific EBV-peptides.³⁵ These tetramers are used in combination with standard flow cytometry and identify cytotoxic T cells as specific for EBV-antigens.³⁴ In addition, anti-EBV tumor function of these cells can be demonstrated using two functional assays used in our lab: 1) ELIspot for gamma-interferon [Enzyme linked immunospot assay] and 2) Chromium release assay. The ELIspot assay measures gamma-interferon, a potent anti-tumor cytokine, released by T cells upon exposure to specific antigen.³⁶ The Chromium assay measures the direct cytolytic activity of these T cells against labeled target.³⁷

Conclusion

An optimal immunogen is prepared when riboflavin is photolyzed in the presence of 10 mM glutathione, a physiological anti-oxidant. The glutathione neutralizes long-lived oxidants produced outside the cell. The cell is now damaged only by photolysis of intracellular riboflavin. Photolysis of intracellular riboflavin induces apoptosis which leads to a more potent immunogen. The process is further enhanced by using synchronized cells to maximize the amount of riboflavin bound to cellular DNA.

In summary we propose to form individualized vaccines by visible light photolysis of aphidocolin synchronized human LCLs transformed by the Epstein-Barr Virus in the presence of riboflavin and glutathione.

LITERATURE CITED

1. Lyons S F, Liebowitz D N. “The roles of human viruses in the pathogenesis of lymphoma.” Semin Oncol. 1998 August; 25(4):461-75.

2.a) Baiocchi R A, Caligiuri M A: “Low dose IL-2 prevents the development of Epstein-Barr virus-associated lymphoproliferative disease in the SCID/SCID mice reconstituted I.P. with EBV-seropositive human peripheral blood lymphoctye.” Proc. Natl. Acad. Sci. USA1994, 91, 5577-5581.

b) Baiocchi R A, Ross M E, Tan J C, Chou C C, Sullivan L, Haldar S, Monne M, Seiden M V, Narula S K, Sklar J, Croce C M, Caligiuri M A: “Lymphomagenesis in the SCID-human mouse involves abundant production of human interleukin-10.” Blood 1995, 85 1063-1074.

c) Baiocchi R A, Khatri V P, Lindemann M J, Ross M E, Papoff G, Caprio A J, Caprio T V, Fenstermaker R, Ruberti G, Bernstein Z P, Caligiuri M A: “Phenotypic and functional analysis of fas (CD95) expression in primary central nervous system lymphoma of patients with acquired immune deficiency syndrome.” Blood 1997, 90, 1737-1746. d) Khatri V J, Baiocchi R A, Bernstein Z P, Caligiuri M A: “Lymphomagenesis in the severe combined immune deficient mouse engrafted with human lymphocytes: clues towards the pathogenesis and treatment of AIDS lymphoma.” International Journal of Oncology 1997 42, 241-246. e) Khatri V P, Baiocchi R A, Peng R, Oberkircher A R, Dolce J M, Ward P M, Herzig G P, Caligiuri M A. “Endogenous CD8+ T-Cell expansion during regression of monoclonal Epstein-Barr Virus-Associated Post-transplant lymphoproliferative disorder. J. Immunol”. 1999, 163, 500-506.

3.a) Rivlin, R. S. “Riboflavin metabolism.” New Engl J. Med. 1970, 283, 463-472.

b) The Merck Index, Twelfth Edition, Budavari, S.; ed. Merck Research Laboratories, 1996, see pages 956, 957 and 1410, and references therein.

4.a) Heelis, P. F. “The photophysical and photochemical properties of flavins (Isoalloxazines)” Chem. Soc. Rev. 1982, 11, 15-39.

b) Heelis, P. F. “The photochemistry of flavins.” Chem. Biochem. Flavoenzymes, 1991, 1, 171-93.

5.a) Metzler, D. E.; Cairns, W. L. “Photochemical degradation of flavins VI.” J. Am. Chem. Soc. 1971, 93, 2772-2777.

b) Song, P.-S.; Metzler, D. E. “Photochemical degradations of flavins IV. Studies of the anaerobic photolysis of riboflavin. IV.” Photochem. Photobiol 1967, 6, 691-709.

6.a) Parks, O. W.; Allen, C. “Photodegradation of riboflavin to lumichrome in milk exposed to sunlight.” J. Dairy Science 1977, 61, 1038-1041.

b) Toyosaki, T.; Hayashi, A. “Structural analysis of the products of milk riboflavin photolysis.” Milchwissenschaft 1993, 48, 607-609.

c) Treadwell, G. E., Jr.; Metzler, D. E. “Photoconversion of riboflavin of lumichrome in plant tissues.” Plant Physio 1972, 49, 991-993.

d) Woodcook, E. A.; Warthesen, J. J.; Labuze, T. P. “Riboflavin photochemical degradation in pasta measured by high performance liquid chromatography.” J. Food. Science 1982, 47, 545-549.

7.a) Tapia, G.; Silva, E. “Photoinduced riboflavin binding to the tryptophan residues of bovine and human serum albumins.” Radiat Environ Biophys 1991, 30, 131-8.

b) Silva, E.; Salim-Hanna, M.; Edwards, A. M.; Becker, M. I.; De Ioannes, A. E. “A light-induced tryptophan-riboflavin binding: biological implications” in nutritional and toxicological consequences of food processing, Friedman, N., ed., Plenum, New York, N.Y., 1991, 33-48.

c) Salim-Hanna, M.; Edwards, A. M.; Silva, E. “Obtention of a photo-induced adduct between a vitamin and an essential amino acid. Binding of riboflavin to tryptophan.” Int. J. Vit. Nutr. Res. 1987, 57, 155-159.

8.a) Broughton, P. M. G.; Rossitor, E. J. R.; Warren, C. B. M.; Gouls, G.; Lord, P. S. “Effect of blue light on hyperbilirubinaemia.” Arch. Dis. Childh. 1963, 40, 666-671.

b) Gromisch, D. S.; Lopez, R.; “Cole, H. S.; Cooperman, J. M. “Light (phototherapy)—induced riboflavin deficiency in the neonate.” J. Ped. 1977, 90, 118-122.

c) Sisson, T. R. C.; Slaven, B.; Hamilton, P. B. “Effect of broad and narrow spectrum fluorescent light on blood constituents.” Birth Defects 1976, XII, 122-133.

d) Sisson, T. R. C. “Photodegradation of riboflavin in neonates.” Fed. Proc 1987, 46, 1883-1885.

9. Crigler, J. F., Jr.; Najjar, V. A. “Congenital familial non-hemolytic jaundice with kemleterus.” Pediatrics 1952, 10, 169-180.

10. Yohannan, M. D.; Terry, H. J.; Littlewood, J. M. “Long term phototherapy in Crigler Najjar syndrome.” Arch. Dis. Child. 1983, 58, 460-462.

11. Olson, J. H.; Hertz, H.; Kjaer, S. K. Bautz, A.; Mellemkjaer, L.; Boice, J. D., Jr. “Childhood leukemia following phototherapy for neonatal hyperbilirubineamia (Denmark).” Cancer Causes and Control 1996, 1, 411-414.

12.a) Burger, P. M.; Simons, J. W. I. M. “Mutagenicity of 8-methoxypsoralen and long-wave ultraviolet irradiation in diploid human skin fibroblasts: An improved risk estimate in photochemotherapy.” Mutat Res 1979, 63, 371-380.

b) Papodopoulu, D.; Averbeck, D. “Genotoxic effects and DNA photoadducts induced in Chinese hamster V79 cells by 5-methoxypsoralen and 8-methoxypsoralen.” Mut Res 1985, 151, 281-291.

c) Cortes, F.; Morgan, W. F.; Varcarel, E. R., et al. “Both cross-links and monoadducts induced in DNA by psoralen can lead to sister chromatid exchange formation.” Exptl Cell Res 1991, 196, 127-130.

d) Studinberg, H. M.; Weller, P. “PUVA, UVA, psoriasis and non-melanoma cancer.” J. Am Acad Dermatol 1993, 29, 1013-1022.

e) Lindelof, B.; Sigurgeirsson, B.; Tegner, E., et al. “PUVA and cancer: A large-scale epidemilogical study.” Lancet 1991, 338, 91-93.

f) Stern, R. S. “Metastatic squamous cell cancer after psoralen photochemotherapy.” Lancet 1994, 344, 1644-5.

g) Lindelof, B.; Sigurgeirsson, B.; Tegner, E., et al. “Comparison of carcinogenic potential of trioxsalen with PUVA and oral methoxsalen PUVA.” Arch Dermatol 1992, 128, 1341-1344.

h) Stern, R. S. and Lange, R. Members of the Photochemotherapy Follow-up Study: Non-melanoma skin cancer occuring in patients treated with PUVA five to ten years after first treatment. J. Invest Dermatol 1988, 91, 120-124.

13. Kuratomi, K.; Kobayashi, Y. “Studies on the Interactions Between DNA and Flavins” Biochim. Biophys. Acta, 1977, 476, 207-217.

14. Dardare, N., Platz, M. S., unpublished research at The Ohio State University.

15. Bensasson, R. V.; Land, E. J.; Truscott, T. G. “Flash photolysis and pulse radiolysis, contributions to the chemistry of biology and medicine,” Pergamon Press, U.K.; 1983.

16. Murov, S. C.; Carmichael, I.; Hug, G. L. “Handbook of Photochemistry” 2nd ed., Marcel Dekker, Inc.; New York, N.Y. 1993.

17.a) Ben-Hur, E. and Horowitz, B. “Advances in photochemical approaches for blood sterilization.” Photochem. Photobiol. 1995, 62, 383-388.

b) Prince, A. M.; Horowitz, B.; Zang, E. M. S. “The development of virus-free labile blood derivatives—a review.” Eur J. Epidermiol 1987, 3, 103-18.

18.a) Brustlein, M.; Knappe, W. R.; Hemmerich, P. “Novel photoalkylation reactions on the flavin nucleus”. Angew Chem Int Ed Engl 1971, 10, 804-806.

b) Knappe, W. R.; Hemmerich, Z. “Covalent intermediates in the flavine-sensitized photodehydrogenation and photodecarboxylation.” Naturforsch, Teil B. 1972, 27, 1011-1032.

19. Sheu, C., Foote, C. S. “Reactivity Toward Singlet Oxygen of a 7,8-Dihydro-8-Oxoguanosine (“8-Hydroxyguanosine”) formed by Photoxidation of a Guanosine Derivative.” J. Am. Chem. Soc. 1995, 117,6439-6442.

20.a) Joshi, P. C. “Ultraviolet radiation-induced photodegradation and ¹O₂, O₂ production by riboflavin, lumichrome and lumiflavin.” Ind. J. Biochem. Biophys 1989, 26, 186-189.

b) Speck, W. T.; Rosenkranz, S.; Rosenkranz, H. S. “Further observations on the photooxidation of DNA in the presence of riboflavin.” Biochim, Biophys Acta 1976, 435, 39-44.

c) Korycka-Dahl, M.; Richardson, T. “Photodegradation of DNA with fluorescent light in the presence of riboflavin and photoprotection by flavin triplet-state quenchers.: Biochim, Biophys Acta 1980, 610, 229-230.

d) Burgstaller, P.; Famulok, M. “Flavin Dependent Photoclevage of RNA at G-U Base Pairs” J. Am. Chem. Soc. 1997, 119(5), 1137-1138.

e) Kochevar, I. E. and Dunn, D. A. “Photosensitized Reactions of DNA: Cleavage and Addition” in Bioorganic Photochemistry Vol. 1, Morrison, H., ed. Wiley, New York, N.Y. 1990, p. 273-316.

f) Joshi, P. C., “Comparison of the DNA Damaging Property of Photosensitized Riboflavin via Singlet Oxygen and Superoxide Mechanisms.” Toxicol. Letters 1985, 26, 211-217.

g) Korycka-Dahl, M., Richardson, T., “Photogeneration of Superoxide Anion upon Illumination of Purines and Pyrimidines in the Presence of Riboflavin: Structure-activity Relationships” J. Food Protection 1980, 43, 19-20.

21. Holmstrom, B. “Spectral studies of the photobleaching of riboflavin phosphate.” Arkiv for kemi 1964, 22, 281-301.

22. Kasai, H., Crain, P. F., Kuchina, Y., Nishimura, S., Outsuyama, A., Tanoka, H., “Formation of 8-hydroxyguanine moiety in cellular DNA by agents producing oxygen radicals and evidence for its repair.” Carcinogenesis 1986, 7, 1847-1851

23. Ennever, J. F., Carr, H. S., Speck, W. T., “Potential for Genetic Damage from Multivitamin Solutions Exposed to Phototherapy Illumination” Pediatr. Res 1983, 17, 192-194.

24.a) Edwards, A. M.; Silva, E.; Jofre, B., Becker, M. I.; de Ioannes, A. E. “Visible light effects on tumoral cells in a culture medium enriched with tryptophan and riboflavin.” J. Photochem. Photobiol B Biol 1994, 24, 179-186.

b) Sato, K.; Taguchi, H.; Maeda, T.; Minami, H.; Asada, Y.; Watanabe, Y.; Yoshikawa, K. “The Primary Cytotoxicity in Ultraviolet A Irradiated Riboflavin Solution is Derived from Hydrogen Peroxide.” J. Invest. Dermatol. 1995, 105, 608-612.

25.a) Ennever, J. F., and Speck, W. T., “Photodynamic Reaction of Riboflavin and Deoxyguanosine” Pediatr. Res 1981, 15, 956-958.

b) Mori, T.; Tano, K.; Takimoto, K.; Utsumi, H. “Formation of 8-hydroxyguanine and 2,6-diamino-4-hydroxy-5-formamidopyrimidine in DNA by Riboflavin Mediated Photosensitization.” Biochem. Biophys Res comm 1988, 242, 98-101.

26. Ennever, J. F.; Speck, W. T. “Photochemical Reactions of Riboflavin: Covalent Binding to DNA and to Poly (dA) Poly (dT)” Pediatr. Res 1983, 17, 234-236.

27.a) Cadet, J. C.; Decarroz, S.; Wang, Y. and Midden, W. R. “Mechanisms and products of photosensitized degradation and related model compounds.” Isr. J. Chem. 1983, 23, 420-429.

b) Raoul, S.; Berger, M.; Buchko, G. W.; Joshi, P. C.; Morin, B.; Weinfeld, M. and Cadet, J. “¹H, ¹³C and ¹⁵N nuclear magnetic resonance analysis and chemical features of the two main radical oxidation products of 2′-deoxyguanosine: oxazolone and imidazolone nucleosides.” J. Chem. Soc. Perkin Trans. 1996, 2 371-381.

c) Buchko, G. W.; Cadet, J.; Morin, B.; Weinfeld, M. “Photooxidation of d(TpG) by riboflavin and methylene blue.” Nucl Acids Res. 1995, 23, 3954-3961.

d) Kasai, H., Yamaizumi, Z., Berger, M., Cadet, J., “Photosensitized Formation of 7,8-Dihydro-8-oxo-2′-deoxyguanosine (8-hudroxy-2′-deoxyguanosine) in DNA by Riboflavin: A Non Singlet Oxygen Mediated Reaction.” J. Am. Chem. Soc. 1992, 114, 9692-9694.

28. Yamamoto, F., Nishimura, S., Kasai, H., “Photosensitized Reactions of 8-hydroxydeoxyguanosine in cellular DNA by Riboflavin.” Biochem. Biophys. Res. Comm. 1992, 187, 809-813.

29. Hoffmann, M. E.; Meneghini, R. “DNA strand breaks in mammalian cells exposed to light in the presence of riboflavin and tryptophan.” Photochem. Photobiol 1979, 29, 299-303.

30. Matherly L H, Schuetz J D, Westin E, Goldman I D. “A method for the synchronization of cultured cells with aphidicolin: application to the large-scale synchronization of L1210 cells and the study of the cell cycle regulation of thymidylate synthase and dihydrofolate reductase.” Anal Biochem. 1989, 182(2), 338-45.

31. Krishan A. “Rapid flow cytofluorometric analysis of mammalian cell cycle by propidium iodide staining.” J. Cell Biol. 1975, 66(1), 188-93.

32. Vermes I, Haanen C, Steffens-Nakken H, Reutelingsperger C. A novel assay for apoptosis. Flow cytometric detection of phosphatidylserine expression on early apoptotic cells using fluorescein labelled Annexin V.” J. Immunol Methods. Jul. 17, 1995;184(1):39-51.

33. Savoldo B, Goss J, Liu Z, Huls M H, Doster S, Gee A P, Brenner M K, Heslop H E, Rooney C M. “Generation of autologous Epstein-Barr virus-specific cytotoxic T cells for adoptive immunotherapy in solid organ transplant recipients. Transplantation 2001 Sep 27;72(6):1078-86.

34. Baiocchi R A, Ward J S, Carrodeguas L, Eisenbeis C F, Peng R, Roychowdhury S, Vourganti S, Sekula T, O'Brien M, Moeschberger M, Caligiuri M A. GM-CSF and IL-2 induce specific cellular immunity and provide protection against Epstein-Barr virus lymphoproliferative disorder. J Clin Invest 2001 September; 108(6):887-94.

35. Altman J D, Moss P A, Goulder P J, Barouch D H, McHeyzer-Williams M G, Bell J I, McMichael A J, Davis M M. Phenotypic analysis of antigen-specific T lymphocytes. Science Oct. 4, 1996;274(5284):94-6.

36. Yang J, Lemas V M, Flinn I W, Krone C, Ambinder R F. “Application of the ELISPOT assay to the characterization of CD8(+) responses to Epstein-Barr virus antigens”. Blood Jan. 1, 2000;95(1):241-8.

37. Coligan J E, Kruisbeek A M, Margulies D H, Shevach E M, Strober W. “Measurement of Cytotoxic NK/LAK Cells.” Current Protocols in Immunology 2001, 7, 7.18.

38. http://www.cobebct.com/library/transfusion/pathogen.html.

39. Platz, M. S.; Goodrich, R. P., Jr. “Isoalloxazine Derivatives to Neutralize Biological Contaminants.” U.S. Pat. No. 6,268,120B1, Jul. 31, 2001 assigned to GAMBRO BCT.

The invention claimed is:

1. A method of producing a cell preparation comprising a plurality of apoptotic Epstein-Barr Virus (EBV)-transformed B lymphocytes, comprising:
a) transforming B lymphocytes with EBV;
b) incubating said EBV-transformed B lymphocytes in a medium comprising a flavin photosensitizer under conditions which permit accumulation of said flavin photosensitizer in said EBV-transformed B lymphocytes;
c) adding a non-toxic antioxidant to said medium; and
d) exposing the EBV-transformed B lymphocytes to photoradiation of an appropriate wavelength to activate said flavin photosensensitizer.

2. The method of claim 1 wherein said flavin photosensitizer comprises riboflavin, a lumichrome (LC)-resistant flavin photosensitizer, or combinations thereof.

3. The method of claim 2 wherein the photosensitzer is riboflavin.

4. The method of claim 2 wherein the LC-resistant flavin photosensitizer has the formula: 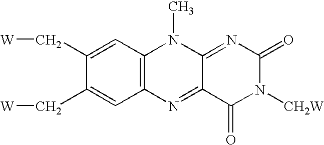
wherein W can be the same or different and is selected from the group consisting of H, ribose, glucose, monoethylene glycol, diethylene glycol, triethylene glycol, and combinations thereof.

5. The method of claim 1 wherein at least 25% of said EBV-transformed lymphocytes are in S phase when said flavin photosensitizer is added to the medium.

6. The method of claim 5 wherein at least 35% of said EBV-transformed lymphocytes are in S phase when said flavin photosensitizer is added to the medium.

7. The method of claim 6 wherein at least 50% of said EBV-transformed lymphocytes are in S phase when said flavin photosensitizer is added to the medium.

8. The method of claim 1 wherein said B lymphocytes are from a human subject.

9. The method of claim 1 wherein said B lymphocytes are from a human subject who is about to undergo an organ transplant.

10. The method of claim 1 wherein said non-toxic antioxidant is glutathione.

11. The method of claim 1 wherein said photoradiation is in the range from about 400 to about 700 nm.

12. The method of claim 11 wherein said radiation is in the range from about 400 to about 500 nm.

13. A cell preparation for eliciting production of EBV-specific T lymphocytes, wherein said cell preparation comprises a plurality of apoptotic EBV-transformed B lymphocytes, wherein said apoptotic EBV-transformed B lymphocytes comprise a DNA-flavin adduct.

14. The cell preparation of claim 13, wherein said cell preparation further comprises an adjuvant.

15. An apoptotic EBV-transformed B lymphocyte, wherein said apoptotic EBV-transformed lymphocyte comprises a DNA-flavin adduct.

16. A method of eliciting production of EBV-specific T cells in a human subject, said method comprising administering a cell preparation to said human subject, said cell preparation comprising a plurality of apoptotic EBV-transformed B lymphocytes, wherein said apoptotic EBV-transformed B lymphocytes comprise a DNA-flavin adduct.

17. The method of claim 16 wherein said cell preparation is partially purified.

18. A method of treating an organ transplant subject, said method comprising administering a cell preparation to said organ transplant subject, said cell preparation comprising a plurality of apoptotic EBV-transformed B lymphocytes, wherein said apoptotic EBV-transformed B lymphocytes comprise a DNA-flavin adduct, wherein said cell preparation is administered in an amount sufficient to elicit production of EBV specific T cells in said organ transplant subject.

19. The method of claim 18 wherein said cell preparation is partially purified prior to administration to said organ transplant subject.

20. The method of claim 18 wherein said organ transplant subject has little to no circulating levels of EBV-specific T cells.

21. The method of claim 18 wherein said organ transplant subject is a human child.

22. The method of claim 18 wherein said cell preparation is administered to said organ transplant subject prior to transplantation and administration of immunosuppressive drugs.

23. The method of claim 18 wherein said cell preparation is administered in an amount sufficient to retard, prevent, or reduce development of post-transplant lymphoproliferative disorder (PTLD) in said organ transplant subject.
